(19) United States
(12) Patent Application Publication
(10) Pub. No.: US 2009/0227860 A1
(43) Pub. Date: Sep. 10, 2009

(54) MR METHOD FOR THE QUANTITATIVE DETERMINATION OF LOCAL RELAXATION TIME VALUES

(75) Inventors: Hannes Dahnke, Hamburg (DE); Tobias Schaeffter, Hamburg (DE)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS, N.V., EINDHOVEN (NL)

(21) Appl. No.: 11/573,408

(22) PCT Filed: Aug. 8, 2005
(86) PCT No.: PCT/IB05/52625

§ 371 Date: Feb. 8, 2007

(30) Foreign Application Priority Data
Aug. 13, 2004 (EP) 04103913.2

(51) Int. Cl. A61B 5/055

(52) U.S. Cl. 600/420

(57) ABSTRACT

The invention relates to an MR method for the quantitative determination of local relaxation time values in an examination volume. Firstly, a plurality of echo signals (1, 2, 3) with different echo time values (t₁, t₂, t₃) are recorded in a phase-sensitive manner. From these echo signals (1, 2, 3), complex MR images (4, 5, 6) are then reconstructed for the different echo time values (t₁, t₂, t₃). Next, local resonant frequency values (7) are calculated for each image point from the echo-time-dependent change in the phases of the complex image values, and then preliminary local magnetic field inhomogeneity values (8) are calculated from the local resonant frequency values (7). The invention proposes that the local relaxation time values (10) be determined from the echo-time-dependent change in the amplitudes of the image values and correction of the local relaxation time values (10) be carried out taking account of final local magnetic field inhomogeneity values. The preliminary magnetic field inhomogeneity values (8) are used as start values for an iterative optimization procedure (19) for determining the final local magnetic field inhomogeneity values.

The invention relates to an MR method for the quantitative determination of local relaxation time values in an examination volume.

The invention furthermore relates to an MR imaging device for carrying out the method and to a computer program for such an MR imaging device.

In MR imaging, as is known, nuclear magnetization within the examination volume of the MR imaging device used is located by means of temporally variable, spatially inhomogeneous magnetic fields (magnetic field gradients). The MR signals used for image reconstruction are usually recorded as a voltage, which is induced in a high-frequency coil arranged in the region of the examination volume, under the effect of a suitable sequence of switched magnetic field gradients and high-frequency pulses in the time domain. A large number of different imaging sequences are known in which, for the purpose of imaging which is as fast as possible, the MR signals are produced as echo signals with different echo time values following excitation of the nuclear magnetization by means of a high-frequency pulse. Such sequences are also referred to as “multiecho sequences”. In this connection, so-called gradient echo sequences, such as the EPI (Echo Planar Imaging) sequence for example, and imaging sequences in which the echo signals are produced by refocusing by means of additional high-frequency pulses, such as the TSE (Turbo Spin Echo) sequence for example, are worth particular mention. The actual image reconstruction from the recorded echo signals usually takes place by Fourier transformation of the time signals. The scanning of the spatial frequency area (so-called “k-space”) assigned to the examination volume, by means of which the field of view (FOV) to be imaged and the image resolution are determined, is defined by the number, the temporal spacing, the duration and the intensity of the magnetic field gradients and high-frequency pulses used. The number of phase encoding steps during scanning of the k-space and thus at the same time the duration of the imaging sequence are defined as a function of the respective requirements in terms of FOV and image resolution.

From the prior art, MR imaging methods are known in which the determination of the local transverse relaxation times of the nuclear magnetization (T₂ or T₂*relaxation) is of particular importance. The visualization and also the quantitative determination of the spatial distribution of the relaxation times are important for example when contrast agents which affect the transverse relaxation of the nuclear magnetization are used in the MR imaging. Such contrast agents, which are based for example on iron oxides, have recently been used also to track marked cells by means of MR and to locate active substances within the examination volume. The spatially resolved determination of transverse relaxation times is also useful in functional MR imaging (fMRI). On the one hand, it is known from the prior art to record T₂*-weighted MR images in order to visualize the spatial distribution of the relaxation times. On the other hand, for some applications, it is desirable to be able to determine the local relaxation times as accurately as possible in quantitative terms. This is the case for example in perfusion studies in which the temporal progress of the passage of a contrast agent bolus through a specific anatomical structure is studied. Another example is the measurement of the dimensions of capillary vessels and the density thereof by means of MR. Quantitative MR relaxometry can also be used for the quantitative determination of the iron content in certain internal organs (e.g. liver, lungs, brain).

One problem with quantitative MR relaxometry is that local inhomogeneities of the static magnetic field shorten the transverse relaxation times of the nuclear magnetization. Such inhomogeneities cannot be avoided particularly in medical MR imaging on account of the different susceptibility properties of the individual patients examined. In medical MR imaging, local inhomogeneities of the magnetic field occur in the region of interfaces between different types of tissue having different susceptibilities. Macroscopic field inhomogeneities are also caused by ferromagnetic objects located in the region of the examination volume. These disruptive influences give rise to accelerated relaxation of the nuclear magnetization. The effect of the field inhomogeneities on the nuclear magnetic relaxation is proportional to the strength of the static magnetic field. In the case of high magnetic field strengths of 3 Tesla or more, as are becoming increasingly customary in medical MR imaging devices, the effects of the field inhomogeneities on the transverse relaxation of the nuclear magnetization can no longer be disregarded. It has been found that, in the case of high magnetic field strengths, the abovementioned susceptibility artifacts lead to completely falsified values when measuring T₂*. The local field inhomogeneities result in a systematic overestimate of the relaxation rate. The consequence may be for example that, on account of the apparently high relaxation rate, the conclusion will be drawn that an iron-oxide-containing contrast agent is present in certain image areas, even though there is actually no contrast agent at the site in question. This therefore results in corresponding cases of misdiagnosis.

Approaches for a solution to the abovementioned problem are already known from the prior art. By way of example, An et al. (Magnetic Resonance in Medicine, volume 47, year of publication 2002, pages 958 to 966) dealt with the spatially resolved measurement of the concentration of deoxyhemoglobin in the brain by means of MR relaxometry. An et al. found that the effects of the local inhomogeneities of the static magnetic field and of deoxyhemoglobin on the transverse T₂* relaxation could be separated from one another, namely on account of the different temporal response of the relaxation components superposed in the recorded MR signals. An et al. propose, in a first step, measuring the local field inhomogeneities by means of highly resolved three-dimensional MR imaging. In a second step, less highly resolved MR data regarding the spatially resolved T₂* measurement are recorded. These data are then corrected according to the previously measured field inhomogeneities, so that the data used for the relaxometry are free of undesirable disruptive influences.

The significant disadvantage of the previously known method is that, on account of the highly resolved three-dimensional imaging which is additionally required, the measurement time is very long overall. The measurement time is more than doubled by the additional image recording step.

Based on this, it is an object of the invention to provide an MR method which allows the quantitative determination of local relaxation time values while eliminating the disruptive influences caused by local field inhomogeneities, wherein the measurement time is to be shorter than in the method known from the prior art.

The invention achieves this object by an MR method having the features as claimed in claim 1.

According to the invention, in a first method step, a plurality of echo signals with different echo time values are recorded in a phase-sensitive manner. The recording of echo signals with different echo time values is necessary in order to be able to analyze the temporal response of the nuclear magnetization to determine the relaxation time values. In the next method step, complex MR images are in each case reconstructed from the echo signals recorded for the different echo time values, so that a complete MR image exists for each echo time value. For each image point of the complex MR images, local resonant frequency values are then calculated, namely by evaluating the echo-time-dependent change in the phases of the complex image values. The phases of the complex image values change in a manner proportional to the echo time, wherein the proportionality factor is in each case the local resonant frequency value. The local resonant frequency value is in turn proportional to the local magnetic field strength. Since, therefore, in this method step the local magnetic field strength is known for each image point, in the next method step a preliminary local magnetic field inhomogeneity value can be calculated for each image point. The local magnetic field inhomogeneity values thus determined are to be regarded as preliminary values since the accuracy with which the local field inhomogeneities are determined in the above-described manner is still not sufficient for the accurate quantitative determination of the local relaxation time values. According to the invention, the local relaxation time values are determined in the last method step from the echo-time-dependent change in the amplitudes of the image values, wherein the local relaxation time values are corrected while taking account of final local magnetic field inhomogeneity values. The final local magnetic field inhomogeneity values are determined using an iterative optimization procedure, wherein the preliminary local magnetic field inhomogeneity values are used as start values. Using the iterative optimization procedure, the previously calculated local magnetic field inhomogeneity values are thus determined more accurately. Here, the optimization procedure makes use of the different temporal response of the amplitudes of the image values, as caused by the nuclear magnetic relaxation and/or the local field inhomogeneities.

The core concept of the invention is to use the information about the local field inhomogeneities which is already present in the recorded image data to save the additional image recording step which is required according to the prior art. This advantageously results in a considerable reduction in measurement time.

The invention is thus based on the knowledge that the course of the static magnetic field in the examination volume can be estimated at least roughly from the phase information contained in the recorded image data. The relaxation time values can then be determined from the echo-time-dependent change in the amplitudes of the image values. A sufficiently accurate determination of the local relaxation time values and of the local field inhomogeneities is then possible purely by means of computer-assisted post-processing of the recorded image data using the iterative optimization procedure. The required calculation time is significantly less than the time required to record additional three-dimensional image data according to the prior art.

Computer-assisted post-processing of the recorded image data in connection with MR relaxometry is already known from the prior art according to Fernández-Seara et al. (Magnetic Resonance in Medicine, volume 44, year of publication 2000, pages 358 to 366). However, in the previously known method, it is not the case that the phase information contained in the image data is used to determine the local field inhomogeneities, as is the essential fundamental idea of the invention, but rather local magnetic field gradient values are estimated and then determined, within the context of an iterative optimization, solely from the temporal response of the amplitudes of the image values. Accordingly, the method according to the invention uses the information contained in the recorded image data in a much more complete and thus more effective manner than is the case in the method known from the prior art. It has been found that, despite this, in terms of calculation time, the method according to the invention is approximately 10 times faster than the method proposed by Fernández-Seara et al.

According to one advantageous embodiment of the method according to the invention, the echo signals are recorded using a slice-selective two-dimensional multiecho sequence for a plurality of image slices which are directly adjacent to one another. Such a multislice image recording provides all the data which are required to calculate the preliminary local magnetic field inhomogeneities as start values for the iterative optimization procedure. The recording of a plurality of image slices which are directly adjacent to one another ensures that the respective preliminary magnetic field inhomogeneity values can be determined with sufficient accuracy for each image point. This can be effected quickly and simply for each image point by interpolation of the local resonant frequency values of the respectively spatially adjacent image points.

When using a multiecho sequence for the phase-sensitive recording of the echo signals, it is also advantageous to record echo signals with the same phase encoding for the different echo time values. When using an EPI sequence, at least some of the so-called “blip” gradients may be omitted in order to achieve this. Overall, of course, the entire k-space must be scanned for each echo time value in order that MR images can in each case be reconstructed for the different echo time values. If echo signals with the same phase encoding exist for different echo time values, this ensures according to the invention that the preliminary local magnetic field inhomogeneity values can be reliably calculated on the basis of the echo-time-dependent change in the phases of the complex image values. For reliable functioning of the method according to the invention, it is specifically advantageous if the complex MR images reconstructed for the different echo time values are recorded using one and the same k-space scanning pattern.

The iterative optimization procedure used according to the invention may comprise the following method steps, which are repeated until a stop criterion is reached: firstly, the echo-time-dependent image values for each image point are corrected according to the corresponding local magnetic field inhomogeneity values. On account of the physical conditions, the echo-time-dependent response of the amplitudes of the image values which is caused by the local magnetic field inhomogeneities is theoretically known. Accordingly, the effects of the magnetic field inhomogeneities can be disregarded from the echo-time-dependent image data. In order to simplify matters, it may be more or less assumed that the local magnetic field course in the region of an individual image point is defined by a linear magnetic field gradient. Local relaxation time values can then be calculated for each image point from the corrected echo-time-dependent image values. This may be effected by adapting the echo-time-dependent image values in each case to a (for example monoexponential) fit function in a conventional manner. This adaptation results in local relaxation time values which represent a first approximation of the actual relaxation time values. Thereafter, an optimization step takes place, said step being designed to determine more accurately the local magnetic field inhomogeneity values, which are at first still preliminary values. This is effected by minimizing the sum of the difference squares of the corrected echo-time-dependent image values from a corresponding relaxation function for each image point, wherein use is made in each case of the previously determined local relaxation time values. In this optimization step, it is assumed that the nuclear magnetic relaxation leads to a given (for example monoexponential) functional dependency of the image values on the echo time. The local magnetic field inhomogeneities give rise to a temporal response of the image values which differs therefrom. This may be used for the above-described optimization procedure in that the local magnetic field inhomogeneity values are optimized in such a way that the correspondingly corrected echo-time-dependent image values approach the relaxation function. The abovementioned steps are then repeated a number of times so that the local relaxation time values and the local magnetic field inhomogeneity values converge iteratively toward the actual values. The iteration takes place until a suitably selected stop criterion is reached.

In order to calculate the local resonant frequency values, it has in practice been found to be advantageous if use is made only of image values the amplitude of which is a predefinable factor (for example ten times) greater than the mean signal noise. This ensures sufficient accuracy of the preliminary local magnetic field gradient values, and calculation time during the determination of the local resonant frequency values is saved by omitting image values with a low signal amplitude.

The method according to the invention is highly suitable for determining the spatial distribution of an iron-oxide-containing contrast agent in the examination volume. The use of small and ultrasmall paramagnetic iron oxide particles (so-called SPIOs) as a contrast agent in MR imaging methods has been of particular interest in recent times. The distribution of these particles in the examination volume is usually assessed on the basis of T₂- or T₂*-weighted MR images. The method according to the invention is particularly suitable for quantitatively determining, by means of MR relaxometry, the local concentration of SPIO particles in the examination volume. Of particular interest is the fact that the SPIO particles of macrophages are recorded. This takes place in the liver following injection of SPIO particles. The SPIO particles may also be used to mark cells (e.g. stem cells) ex vivo. By virtue of the quantitative determination of local relaxation time values according to the invention, such marked cells can then be tracked following injection into the body of a patient. The method according to the invention advantageously makes it possible to distinguish SPIO particles taken up by cells from SPIO particles located outside cells, based on the differences of T₂ and T₂*.

In order to carry out the method according to the invention, use may be made of an MR imaging device comprising recording means for recording echo signals, and computer means for the quantitative determination of local relaxation time values from the echo signals. The above-described method can be carried out on the MR imaging device according to the invention by means of suitable program control of the computer means. The method according to the invention may be made available to users of MR imaging devices in the form of a corresponding computer program. The computer program may be stored on suitable data carriers, such as CD-ROMs or floppy disks for example, or it may be downloaded from the Internet onto the computer means of the MR imaging device.

The invention will be further described with reference to examples of embodiments shown in the drawings to which, however, the invention is not restricted.

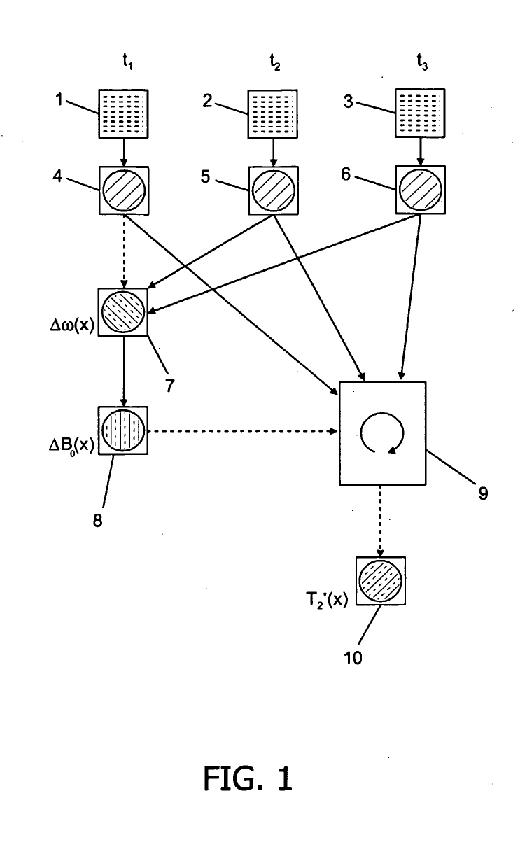

FIG. 1 schematically shows the progress of the method according to the invention.

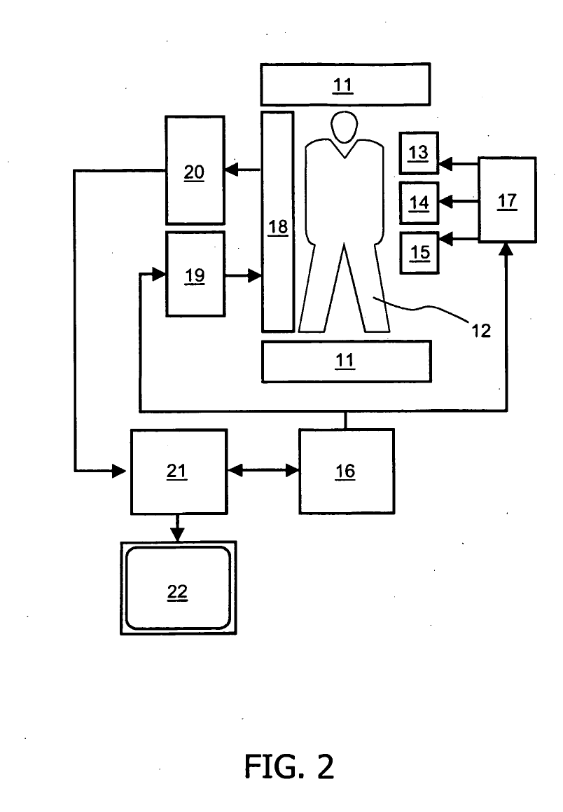

FIG. 2 shows an MR device according to the invention.

The method shown in FIG. 1 begins with the phase-sensitive recording of a plurality of echo signals with three different echo time values t₁, t₂ and t₃. A data record 1, 2 and 3 exists for each of these echo time values. In each case, complex MR images 4, 5 and 6 are reconstructed from the three data records 1, 2 and 3. An MR image 4, 5 and 6 thus exists for each echo time value t₁, t₂ and t₃. For each image point of the MR images 4, 5 and 6, local resonant frequency values are calculated from the echo-time-dependent change in the phases of the complex image values. The result is a data record 7 which comprises the local resonant frequency values as frequency shift values Δω(x) for each image point. Preliminary local magnetic field inhomogeneity values, once again for each image point, are then calculated from the data record 7. In the example of embodiment, the local magnetic field inhomogeneity values exist as ΔB₀(x), that is to say as magnetic field differences between respectively spatially adjacent image points. Finally, the MR images 4, 5 and 6 and the preliminary local magnetic field inhomogeneity values 8 are fed to an iterative optimization algorithm 9 as input data. Here, the local relaxation time values are determined from the echo-time-dependent change in the amplitudes of the image values of the MR images 4, 5 and 6, wherein the local relaxation time values are corrected taking into account final magnetic field inhomogeneity values. For the iterative optimization procedure used, the preliminary local magnetic field inhomogeneity values according to the data record 8 are used as start values. The local relaxation time values T₂*(x) exist at the end as data record 10.

The iterative optimization procedure for determining the final local magnetic field inhomogeneity values may be implemented as follows:

Firstly, the echo-time-dependent image values S(TE) for each image point are corrected according to the corresponding local magnetic field gradient values ΔB₀, and specifically according to the following formula:

$${S}_{0} \cdot exp ( - \frac{TE}{{T}_{2}^{*}} ) = S ( TE ) / sinc ( γΔ {B}_{0} / 2 \cdot TE )$$

Here, S₀ is the absolute value of the image value amplitude. This value is of no further interest. TE is the respective echo time value. T₂* is the actual local transverse relaxation time of interest. S(TE) is the echo-time-dependent change in the image value amplitude. γ is the gyromagnetic ratio. The correction thus takes place by dividing the echo-time-dependent image values by the value of a sinc function, which depends on the local magnetic field gradient value ΔB₀ and on the echo time TE. The sinc function represents the temporal response of the image value amplitude, which results from the effect of the magnetic field gradient value ΔB₀. The local relaxation time T₂* can then be determined from the image values thus corrected, by adaptation to an exponential function. In the next step, the sum of the difference squares SD is calculated according to the following formula:

$$SD = \sqrt{\frac{{\sum }_{i}^{n} {( {S}_{0} exp ( - \frac{{TE}_{i}}{{T}_{2}^{*}} ) - \frac{S ( {TE}_{i} )}{sinc ( γΔ {B}_{0} / 2 \cdot {TE}_{i} )} )}^{2}}{n - 1}}$$

Summation is carried out over all the echo time values TEᵢ. The local magnetic field gradient value ΔB₀ is optimized for the relevant image point by minimizing the above sum of the difference squares. An attempt is thereby made to make the corrected echo-time-dependent image values coincide as far as possible with a monoexponential relaxation function. Once an optimized local magnetic field gradient value has been found, the correction of the echo-time-dependent image values is repeated using the optimized local magnetic field gradient value, and an improved relaxation time value T₂* is determined. The overall procedure is repeated until convergence can be ascertained both in terms of the local magnetic field gradient value ΔB₀ and in terms of the local relaxation time value T₂*.

FIG. 2 shows a block diagram of an MR imaging device on which the method according to the invention can be carried out. The MR imaging device consists of a main field coil 11 for generating a homogeneous static magnetic field in an examination volume in which a patient 12 is located. The MR imaging device furthermore has gradient coils 13, 14 and 15 for generating magnetic field gradients in different spatial directions within the examination volume. The temporal and spatial course of the magnetic field gradients within the examination volume is controlled by means of a central control unit 16, which is connected to the gradient coils 13, 14 and 15 via a gradient amplifier 17. The MR imaging device shown also comprises a high-frequency coil 18 for generating high-frequency fields in the examination volume and for receiving echo signals from the examination volume. The high-frequency coil 18 is connected to the control unit 16 via a transmitter unit 19. The echo signals recorded by the high-frequency coil 18 are demodulated and amplified by a receiver unit 20 and fed to a reconstruction and visualization unit 21. The high-frequency coil 18 together with the receiver unit 20 forms the recording means of the MR imaging device. The control unit 16 and the reconstruction and visualization unit 21 are the computer means of the MR imaging device according to the invention. The echo signals processed by the reconstruction and visualization unit 21 can be displayed on a screen 22. The reconstruction and visualization unit 21 and the control unit 16 have suitable program control for carrying out the method according to the invention.

1. An MR method for the quantitative determination of local relaxation time values (T₂*) in an examination volume, comprising the following method steps:
a) phase-sensitive recording of a plurality of echo signals (1, 2, 3) with different echo time values (t₁, t₂, t₃);
b) reconstruction of complex MR images (4, 5, 6) from the echo signals (1, 2, 3) for the different echo time values (t₁, t₂, t₃);
c) calculation of local resonant frequency values (7) for each image point from the echo-time-dependent change in the phases of the complex image values;
d) calculation of preliminary local magnetic field inhomogeneity values (8) from the local resonant frequency values (7);
e) determination of the local relaxation time values (10) from the echo-time-dependent change in the amplitudes of the image values and correction of the local relaxation time values (10) taking account of final local magnetic field inhomogeneity values, wherein the preliminary magnetic field inhomogeneity values (8) are used as start values for an iterative optimization procedure (19) for determining the final local magnetic field inhomogeneity values.

2. A method as claimed in claim 1, wherein the echo signals are recorded using a slice-selective two-dimensional multiecho sequence for a plurality of image slices which are directly adjacent to one another.

3. A method as claimed in claim 2, wherein echo signals with the same phase encoding are recorded for different echo time values.

4. A method as claimed in claim 2, wherein the multiecho sequence is an EPI sequence.

5. A method as claimed in claim 1, wherein the iterative optimization procedure comprises the following method steps, which are repeated until a stop criterion is reached:
correction of the echo-time-dependent image values for each image point according to the corresponding local magnetic inhomogeneity values;
calculation of local relaxation time values for each image point from the corrected image values;
optimization of the local magnetic field gradient values by minimizing the sum of the difference squares of the corrected echo-time-dependent image values from a relaxation function for each image point.

6. A method as claimed in claim 1, wherein, in order to calculate the local resonant frequency values, use is made only of image values the amplitude of which is a predefinable factor greater than the mean signal noise.

7. The use of a method as claimed in claim 1 for determining the spatial distribution of an iron-oxide-containing contrast agent in the examination volume.

8. An MR imaging device comprising recording means (18, 20) for recording echo signals, and computer means (16, 21) for the quantitative determination of local relaxation time values (T₂*) from the echo signals, wherein the computer means (16, 21) are designed, by means of suitable program control, to carry out the method as claimed in claim 1.

9. A computer program for an MR imaging device, wherein a method as claimed in claim 1 is implemented by the computer program on computer means of the MR imaging device.
